(19) United States
(12) Patent Application Publication
(10) Pub. No.: US 2008/0027461 A1
(43) Pub. Date: Jan. 31, 2008

(54) Intraocular Lens Inserter Plunger

(76) Inventors: Edward A. Vaquero, Fairport, NY (US); George E. Diaz, Rochester, NY (US); Jon P. Cullen, Churchville, NY (US); Benoit Bessiere, Plaisance due Touch (FR)

Correspondence Address: Bausch & Lomb Incorporated, One Bausch & Lomb Place, Rochester, NY, 14604-2701, US

(21) Appl. No.: 11/782,324

(22) Filed: Jul. 24, 2007

Related U.S. Application Data
(60) Provisional application No. 60/833,203, filed on Jul. 25, 2006.

(51) Int. Cl. A61F 9/007

(52) U.S. Cl. 606/107

(57) ABSTRACT

An IOL injector, comprising an injector body; and a plunger. The plunger comprises a shaft comprising a moldable material having a flexural modulus greater than 600 thousand psi, and a plunger tip. The moldable material may have a flexural modulus greater than 750 thousand psi or greater than 1 million psi. The plunger shaft may comprise, for example, Polybutylene Terephthalate (PBT), Polyphthalamide (PPA) or liquid crystal polymer (LCP).

### CROSS-REFERENCE

This application claims the benefit of Provisional Patent Application No. 60/833,203 filed Jul. 25, 2006.

### FIELD OF INVENTION

The present invention relates to injectors for intraocular lenses, and more particularly to plungers for such injectors.

### BACKGROUND OF THE INVENTION

IOLs are artificial lenses used to replace natural crystalline lenses of patients' when their natural lenses are diseased or otherwise impaired. IOLs come in a variety of configurations and materials.

Techniques for implanting such IOLs in an eye are known. Typically, such techniques include making an incision in a patient's cornea and inserting an IOL into the patient's eye through the incision. In one technique, a surgeon uses surgical forceps to grasp the IOL and insert the IOL through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+ mm) since smaller incisions have been attributed with reduced post-surgical healing time and reduced complications such as induced astigmatism.

Benefits have been attained from molding injector devices from disposable materials (e.g., a moldable polymer). For example, such devices have been determined to be more economically manufactured in addition to being more conducive to maintaining sterility during operations.

In order for an IOL to fit through a small incision, it is typically folded and/or compressed prior to entering the eye where it will assume its original unfolded/uncompressed shape. To fold and/or compress and IOL, an injector is provided with a funnel-shaped compression chamber. It is desirable that an IOL be expelled from the tip of the IOL injector and into the eye in an undamaged condition and in a uniform and consistent manner. One aspect of uniform and consistent delivery is delivery in a predictable orientation.

Should an IOL be damaged or expelled from the injector in an incorrect orientation, a surgeon may need to remove or further manipulate the IOL in the eye, possibly resulting in trauma to the surrounding tissues of the eye. To date, techniques to ensure proper insertion have included, for example, specialized IOL loading apparatus, injectors having specialized lumen shapes, yet there remains a problem of lens rotation. There are particular problems arising as the incision size decreases and the amount of compression and/or folding of the IOL is increased.

### SUMMARY

Aspects of the present invention apply a recognition that the delivery of a lens in a proper orientation can be substantially improved by providing a molded injector with a moldable plunger that is more rigid than conventional molded polymeric plungers. The improvements are particularly significant for inserting a lens when the lens is to be highly compressed in a compression chamber, such as is typically done before insertion into a relatively small incision.

It has been found that such plungers provide a more predictable source of force for advancing a lens through an injector lumen thereby reducing the likelihood of lens rotation as the lens is pushed through the injector lumen, including through the compression chamber of an injector.

An aspect of the invention is directed to an IOL injector, comprising an injector body; and a plunger, where the plunger comprises a shaft comprising a moldable polymeric material having a flexural modulus greater than 600 thousand pounds per square inch (psi), and a plunger tip. The moldable polymeric material may have a flexural modulus greater than 750 thousand psi or greater than 1 million psi. The plunger shaft may comprise, for example, Polybutylene Terephthalate (PBT), Polyphthalamide (PPA) or liquid crystal polymer (LCP).

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a projection view of an injector according to aspects of the present invention;

FIG. 2 is a projection view of an injector plunger according to aspects of the present invention; and

FIG. 3 is an expanded view of an example of a plunger tip.

### DETAILED DESCRIPTION

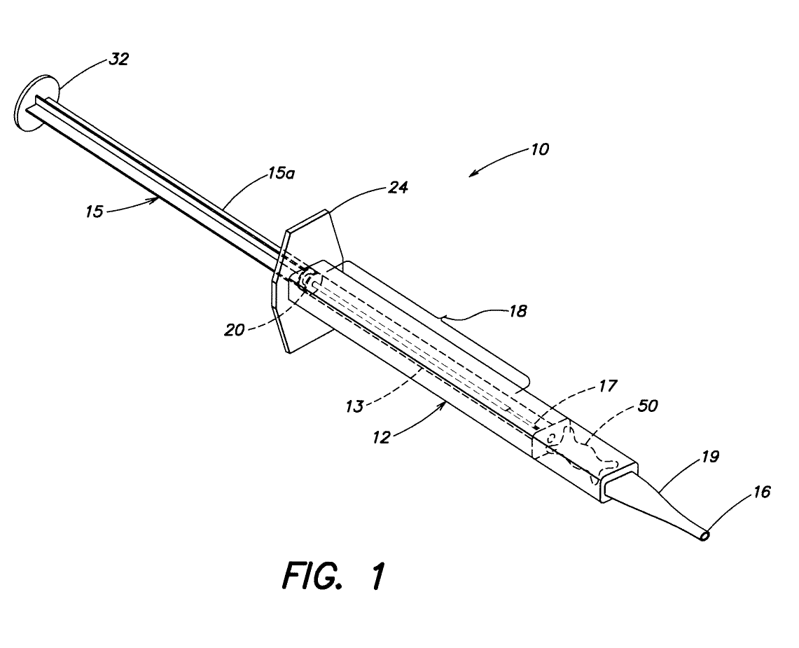

FIG. 1 is a projection view of an example of an IOL injector device 10 according to aspects of the present invention. Injector device 10 includes an injector body 12 defining a longitudinal passageway (i.e., lumen 13) extending from a proximal end of the injector to a distal tip of the injector. Substantial compression of an IOL may be achieved by the sloped surfaces of compression chamber 19. The injector includes a plunger 15 which telescopes in the proximal end of the tubular body, a distal tip 16 configured for insertion into an incision in an eye for passing an IOL from the injector into the eye.

A simplified IOL 50 is illustrated in body 12. It is to be appreciated that injector 10 may be provided with any suitable IOL loading apparatus such a hinged door in the side of the injector body. The proximal end of the body includes a finger hold 24 and plunger 15 includes a finger press 32 at the proximal end thereof for pressing and advancing the plunger through lumen 13 in the manner of a syringe. A plunger tip 17 is provided at the distal end of the plunger for engaging and pushing IOL 50 through and out of tip 16 of the injector body 12.

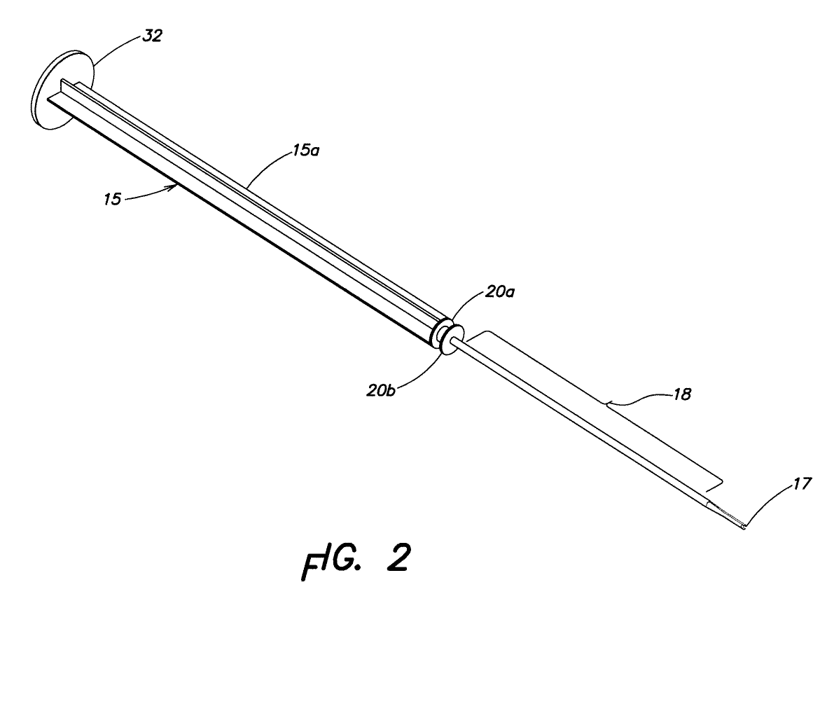

FIG. 2 is a projection view illustrating further details of plunger 15. According to aspects of the present invention, the plunger is provided with a rigid shaft 18. As stated above plunger 15, comprising a rigid shaft, advances an IOL (by action of the tip) in a more stable manner (i.e., rotation of the lens is reduced or substantially eliminated).

According to aspects of the present invention, a plunger having a shaft comprising a polymeric material that has a flexural modulus of at least 600 thousand pounds per square inch (kpsi) is provided. In some embodiments, the flexural modulus is greater than 750 thousand psi, and in some embodiments greater than 1 million psi (Mpsi).

Provided below are examples of families of polymeric materials that provide flexural moduluses of at least 600 kpsi It is to be appreciated that a given material may provide a suitable modulus either “neat” (i.e., without fillers), filled (e.g., with glass fiber or glass beed or carbon fiber or mineral fillers at a suitable percentage), or including copolymers of multiple polymers. The examples of families of materials include—

Acrylonitrile Butadiene Stryene (ABS)

Acetal

Acrylic

Polyketone

Polyetheretherketone (PEEK)

Liquid Crystal Polymer (LCP)

Nylon—various permutations (46,6,66/6,66,610,612,11,12),

Polyamide

Polyimide

Polyarylamide

Polycarbonate

Polybutylene Terephthalate (PBT)

Polyethylene Terephthalate (PET)

Polyester

Polyetherimide

Polyethersulfone (PES)

Polyimide, Polyphenylene Ether

Polyphenylene, Sulfide (PPS)

Polyphthalamide (PPA)

Polypropylene

Polystyrene

Polysulfone

Polyphenylsulfone

Polyarylsulfone

Polyvynalchloride

Polyphenyloxide (PPO)

SAN

In some embodiments, it is desirable that the plunger 15 be properly aligned and rotationally fixed within the lumen 13 so as to further ensure proper engagement of the IOL 50 with plunger tip 17 and to reduce the likelihood of rotation of a lens. Plunger alignment and rotational fixing can be achieved, for example, by forming the body 12 and proximal end 15a of the plunger in a non-circular (e.g., oval) cross-sectional shape.

Additionally, to assist in controlling forward movement of the plunger within the body, a plunger may use one or more rubber O-rings or other molded alignment features 20a, 20b which provide friction between the plunger and inside wall of the body.

It is to be understood that aspects of the invention are directed to the plunger shaft. In embodiments of the invention that are according to such aspects, other parts of the injector (e.g., the plunger tip, the cross-sectional shape of the injector body, the IOL loading area, the injector distal tip, etc.) may be of any desired configuration.

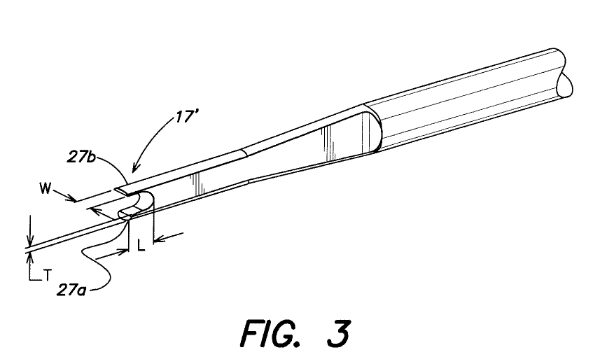

It is to be appreciated that a soft tip (e.g., comprising silicone) may be provided as the plunger tip, for example, to protect an IOL from the material comprising the shaft. Such a configuration is particularly advantageous, in the embodiments where the high flexural modulus material comprising the shaft has a propensity to damage the lens (e.g., due to jagged edges perhaps caused by a filler). A soft tip may be applied, for example, by dip coating the distal end of the plunger in a soft material or by forming the soft material such that it has a sleeve in the proximal end of the tip, where the distal end of the plunger can be received. For example, a soft tip as described in U.S. Pat. No. 5,735,858, to Makker may be used. A tip (regardless of whether it is soft or integrated with the plunger shaft) may be selected to have any suitable shape. For example, a tip can have forked shape (as illustrated in FIG. 3) or a distal end having a convex shape.

### EXAMPLE #1

One example of a material that has been found to be suitable for formation of a plunger shaft is Polyphthalamide (PPA) having a 40% mineral fill (e.g., calcium carbonate fill). PPA having a 40% fill was measured to have a flexural modulus of 1.1 Mpsi using ASTM D790 or 1.0 at 23° C. using ISO 178. For example, PPA is available from Solvay Plastics of Brussels, Belgium.

In addition to the suitable flexural modulus provided by PPA, a desirable characteristic of PPA is that, upon curing, a resultant plunger is free of sharp edges that may cause tearing of an IOL. Also it was found that PPA provides advantageous moldability characteristics that permit small features to be manufactured. Accordingly, in some embodiments, the entire plunger (including the plunger tip) is made of PPA (i.e., the tip is made of the same material as the shaft and may be integrated therewith). Accordingly, in such embodiments a plunger may be used without a soft tip. For example, PPA has been demonstrated to fill a mold of a forked tip. FIG. 3 shows an example of such a forked tip 17′. For example, the prongs 27a, 27b of the fork may have a length L of approximately 0.9 mm, a thickness T of approximately 0.25 mm, and a width W of approximately 7.5 mm. To manufacture such a tip, it may be advantageous if a vent is provided at the tip-forming portion of a mold cavity.

### EXAMPLE #2

Another example of a material that has been found to be suitable for formation of a plunger shaft is Polybutylene Terephthalate (PBT) having a 30% glass fill. PBT having a 30% glass fill was measured to have a flexural modulus of 1.1 Mpsi using ASTM D790. For example, such PBT is available from General Electric Corporation of Fairfield, Conn. under the tradename, Valox®. Valox having a glass fill, while having a suitable flexural modulus was found to have sharp edges that may cause damage to IOLs. Accordingly, it may be desirable to add a soft tip as described above, over the distal end of the shaft. For example, the glass fill may comprise glass fiber particles having a cylindrical shape with an average length of approximately 3 mm.

### EXAMPLE #3

Yet another example of a material that has been found suitable for formation of a plunger shaft is Polyarylamide (PAA) having a 50% glass fill (the fill particles having a diameter of approximately 10 to 15 microns and a length of approximately 1.6 mm). For example, such PAA is available from Solvay Plastics under the tradename IXEF 1022. It was found that PPA provides advantageous moldability characteristics that permit small features to be manufactured. For example, PAA has been demonstrated to fill a mold of a forked tip. Additionally, PAA forms a stratified structure upon molding such that the glass fill is covered by a skin of polymer material which reduces the amount of glass fiber on the surface of a molded part.

In some embodiments, the entire plunger (including the plunger tip) is made of PAA (i.e., the tip is made of the same material as the shaft and may be integrated therewith). In such embodiments a plunger may be used without a soft tip.

Other examples of materials having suitable flexural moduluses are PBT having a 30% glass fill, available from General Electric, under the name Valox 420 HP; LCP having a 15% mineral fill, available from Ticona division of Celanese Inc. of Auburn Hills, Mich., under the name Vectra A515; LCP having a 30% mineral fill, available from Ticona, under the name Vectra E530i; LCP having a 30% mineral fill, available from Dupont Inc, under the name Zenite 6330 NC 010.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An IOL injector, comprising:
(A.) an injector body; and
(B.) a plunger, comprising
(i.) a shaft comprising a polymeric material having a flexural modulus greater than 600 thousand psi, and
(ii.) a plunger tip.

2. The IOL injector of claim 1, wherein the polymeric material has a flexural modulus greater than 750 thousand psi.

3. The IOL injector of claim 1, wherein the polymeric material has a flexural modulus greater than 1 million psi.

4. The IOL injector of claim 1, further comprising a soft tip disposed at a distal end of the plunger.

5. The IOL injector of claim 1, wherein the plunger shaft comprises Polybutylene Terephthalate (PBT).

6. The IOL injector of claim 1, wherein the plunger shaft comprises Polyphthalamide (PPA).

7. The IOL injector of claim 1, wherein the plunger shaft comprises liquid crystal polymer (LCP).

8. The IOL injector of claim 1, wherein the plunger shaft comprises polyarylamide (PAA).
